(19) United States
(12) Patent Application Publication
(10) Pub. No.: US 2026/0076876 A1
(43) Pub. Date: Mar. 19, 2026

(54) A TEAT PRODUCT AND A METHOD FOR PRODUCING A TEAT PRODUCT

(71) Applicant: Rosemary Mobbs

(72) Inventor: Rosemary Mobbs

(21) Appl. No.: 19/106,437

(22) PCT Filed: Aug. 25, 2023
(86) PCT No.: PCT/AU2023/050820

§ 371 Date: Feb. 25, 2025

(30) Foreign Application Priority Data
Aug. 25, 2022 (AU) 2022902435

(51) Int. Cl. A61J 13/00

(52) U.S. Cl. CPC A61J 13/00

(57) ABSTRACT

A teat product having a base portion and a nipple portion extending from the base portion, the teat product having a pliable region, wherein said pliable region of the teat product is configured to be deformable in shape in response to force applied by a mouth of a feeding infant.

### FIELD OF THE INVENTION

The present disclosure relates to a maternity teat product for use with baby bottles, and/or as soothers and nipple shields, and a method for producing the teat product.

### BACKGROUND OF THE INVENTION

A number of products exist to assist new mothers with infants that involve the use of an imitation teat designed to imitate the nipple of the mother's breast. Such teat products include baby bottle teats, soothers, and nipple shields.

Baby bottles are commonly used to feed infants and toddlers in lieu of breast feeding and comprise a bottle with a teat, typically made of silicone, latex, or rubber. Baby bottles are commonly used to feed infants a range of liquids, such as expressed breast milk, milk made from evaporated milk formula, or paediatric electrolyte solution. Infants may need to be bottle-fed for many different reasons, such as simple convenience, insufficient breast milk supply, nipple damage or mastitis.

Infant soothers (or pacifiers) are used to calm infants by providing an imitation teat for the infant to suck on, which simulates the act of breast feeding and can help calm an upset infant. The soother typically has a silicone, rubber or plastic teat attached to a mouth shield and handle.

Nipple shields are used to protect a feeding mother's nipples during breast feeding. They consist of a soft, thin, nipple-shaped sheath that partially covers the breast, including covering the areola and nipple, which helps protect against cracked and sore nipples whilst still allowing the infant to breastfeed. Use of a nipple shield can also assist with developing an infant's latching technique.

Use of imitation teats in baby bottles, soothers, nipple shields and other imitation teat products can result in an infant developing “nipple confusion” due to different nipple shapes in the baby's mouth. This can result in an infant being reluctant to breastfeed, and prefer bottle-fed milk. With increased use of baby bottles and other imitation teats, nipple confusion is becoming more and more prevalent and is having a significant impact on the rate of breastfeeding amongst new mothers.

Breastfeeding is widely acknowledged as providing substantial health benefits for infants and any decline in the rate of breastfeeding amongst new mothers results in a consequent increase in the prevalence of certain health complications and allergies.

It is desirable to provide a teat product that substantially overcomes or at least ameliorates one or more of the above disadvantages, or to provide a useful alternative to known teat products.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention provides a teat product having a base portion and a nipple portion extending from the base portion, the teat product having a pliable region, wherein said pliable region of the teat product is configured to be deformable in shape in response to force applied by a mouth of a feeding infant.

Advantageously, the present invention provides a teat product that provides an infant with a better feeding experience, which is closer to a regular breastfeeding experience. This improved experience is due in part to the provision of the pliable region, which provides the teat product with a degree of deformability and flexibility in shape in response to force (e.g. suction/vacuum created by suction) applied by the mouth of the feeding infant. Prior art teat products are conventionally relatively rigid and inflexible products that require the infant to continuously overcome an inherent bias in the teat product structure during each application of force and provide minimal deformability in product shape. In contrast, the present invention provides a more readily deformable, flexible and responsive teat product due to the improved flexibility and deformability of the pliable region of the teat product.

It will be appreciated that whilst it is envisaged that the infant and mother are human mammals, the invention may be suitably employed for other mammals.

In an embodiment, the teat product is further configured to be deformable in shape to adapt at least in part to movement in a nipple (e.g. elongation). Preferably, said pliable region provides, at least in part, said teat product with said deformability in shape.

Preferably, the pliable region of the teat product is configured to have increased stretchability relative to a remainder of the teat product. The pliable region is preferably resilient, resulting in the pliable region springing back into its original shape after deformation (e.g. after elongation when being stretched). The pliable region of the teat product may be configured to enable elongation and/or deformation of the nipple portion.

In an embodiment, the nipple portion is disposed in the pliable region. In an embodiment, the pliable region may include at least a portion of the nipple portion.

In an embodiment, the pliable region is configured to enable at least a portion of the teat product to substantially conform in shape to a nipple. In other words, references to deformability herein can include conformability of the teat product. In an embodiment, the pliable region is configured to enable the nipple portion to substantially conform in shape to a nipple. Preferably, the nipple portion is disposed in the pliable region, and the nipple portion is configured to substantially conform in shape to a nipple. Advantageously, by being conformable in shape to the nipple of the mother, the teat product helps reduce nipple confusion by providing the infant with a familiar nipple shape in the infant's mouth (i.e. the mother's nipple). For example, the nipple portion is configured to conform in shape to the nipple when the infant imprints or otherwise impresses on the nipple portion. This is particularly important during the infant's latchment stage (typically the first six months after birth), which is the first emotional stage of development during which the infant recognises its mother through the oral tactile perception of the nipple in their mouth. Further, the closeness of the nipple to the teat product can improve nipple stimulation, thereby improving the release of lactation hormones. This can result in improved milk supply to the infant relative to large, non-deformable and more rigid teat products.

In an embodiment, the pliable region is formed of a different material to the remainder of the teat product. In another embodiment, the pliable region is formed of the same material as to the remainder of the teat product. In an embodiment, the pliable region is defined by an altered region of said teat product, said altered region providing said deformability of the teat product. Said altered region may have a reduced thickness to the remainder of the teat product. Said altered region may include a textured surface. Said altered region may include one or more recesses or grooves, said one or more recesses or grooves enabling, at least in part, said deformability in shape.

In an embodiment, the pliable region is formed of a mesh. As used herein, reference to a mesh or a meshed region is to be understood as a reference to a region having a plurality of openings, be said openings formed through perforation or formed between a network of interlinked portions. In an embodiment, the pliable region includes a plurality of openings, wherein said plurality of openings are configured to impart said deformability in shape of the pliable region. Advantageously, said plurality of openings provide the teat product with the desired responsiveness (deformability and stretchability), such as when the nipple portion elongates during suckling/breastfeeding. Further, the plurality of openings support the nipple portion in being conformable in shape to a nipple of the mother, thereby helping to reduce nipple confusion. Further, the openings prevent or reduce milk from pooling in the base of the teat product, a known issue of existing teat products.

In an embodiment, the openings may have a diameter between about 0.1 mm and about 3 mm, preferably between 1 mm and 3 mm. The openings may be substantially circular in shape. However, in other embodiments the openings may be of a non-circular shape. In an embodiment, each of the plurality of openings are of the same size. In another embodiment, the plurality of openings may be of different size. For example, openings towards a centre of the pliable region may be smaller than openings peripheral to said centre. It will be appreciated that the size and/or shape of the openings influence in part the deformability of the pliable region of the teat product. In an embodiment, the pliable region may be formed by producing a plurality of closely spaced openings in a region of the teat product. Spacing between openings of the pliable region may range between about 0.1 mm and about 3 mm. Although other suitable opening spacing can be utilised.

In an embodiment, the nipple portion is arranged substantially centrally of the pliable region.

In an embodiment, the nipple portion is arranged substantially centrally of the base portion.

Preferably, the pliable region is coaxial with the nipple portion.

In an embodiment, the pliable region is between about 0.5 cm and 5 cm in length or diameter, preferably between about 1 cm and 5 cm, and further preferably between 2 cm and 5 cm. In an embodiment, substantially the whole teat product is contained within the pliable region.

In an embodiment, the teat product may include a plurality of separate pliable regions. The plurality of pliable regions may be arranged in any suitable manner to confer the desired deformability, flexibility and/or stretchability of the teat product. In one example, the plurality of pliable regions may be arranged in a pattern about the base portion. In another example, the plurality of pliable regions may be arranged in a pattern about the nipple portion. The plurality of pliable regions may be substantially identical in overall shape. Alternatively, the plurality of pliable regions may not all share the same overall shape. Each of the plurality of pliable regions may include substantially the same pattern and/or opening size. Alternatively, at least one pliable region of the plurality of pliable regions may be formed of a different pattern and/or opening size.

In an embodiment, the pliable region may include a pliable transition portion between the base portion and the nipple portion. Said transition portion may define a ring of pliable material at an inner end of the nipple portion. Advantageously, said pliable transition portion enables the nipple portion to be stretchable away from the base portion.

In an embodiment, fluid is communicated through the pliable region. For example, fluid is communicated through the plurality of openings. Alternatively, the teat product may include one or more additional openings, separate to the plurality of openings of the pliable region, disposed in the nipple portion, said one or more additional openings configured to communicate fluid therethrough.

In an embodiment where the pliable region is formed of a mesh, the mesh may be a woven mesh. In another embodiment, the mesh may be a non-woven mesh. In an embodiment, the mesh may be in the form of netting. In an embodiment, the mesh may be in the form of chainmail mesh.

In an embodiment, the teat product may include medicament interspersed in openings of the pliable region. In this way, the teat product can be used to help administer a suitable dosage of the medicament to the infant.

The teat product may be formed of any suitable material to achieve the desired deformability and flexibility. Preferably, the teat product is formed of a silicone material, such as medical grade silicone. However, in other embodiments, latex, rubber, or similar may be used. The teat product may be formed of a bio-compatible material. The teat product may be formed of a smart material. In a preferable embodiment, the combination of the plurality of openings and the suitable material forming the teat product (or of the pliable region thereof) provides added deformability and stretchability relative to a teat product formed only of the suitable material.

The teat product may be a baby bottle teat, a soother, or a nipple shield.

The teat product may be formed in a moulding process. For example, the teat product may be formed in an injection moulding process. However, it will be appreciated that other manufacturing techniques can be used.

In an embodiment where the teat product is a nipple shield, the teat product may include at least one cut-out portion configured to facilitate direct contact of the infant with the mother during feeding. For example, the at least one cut-out portion may facilitate direct contact between the infant's cheek and the mother's breast. The at least one cut-out portion is preferably substantially semi-circular in shape. However, the at least one cut-out portion may be of any other suitable shape to facilitate direct contact of the infant with the mother during feeding in a manner to enhance the feeding experience for the infant. In an embodiment, the teat product includes two cut-out portions.

In an embodiment, the teat product may have a thickness of between about 0.1 mm and 4 mm, preferably between 0.2 mm and 3 mm. In an embodiment, the teat product may have a substantially uniform thickness. In another embodiment, the teat product may have different thickness throughout. In an embodiment, the base portion includes a first thickness, and the nipple portion includes a second thickness, wherein the first thickness is greater than the second thickness. In such an embodiment, the first thickness may gradually taper to said second thickness, or there may be a step change in thickness. In an embodiment, the base portion includes a first thickness, and the nipple portion includes a second thickness, and a transition portion, between the base portion and nipple portion, includes a third thickness, wherein the first thickness is greater than the second thickness, and the second thickness is greater than the third thickness. In such an embodiment, the first thickness may gradually taper to said third thickness and then gradually thicken to said second thickness, or there may be a step change in thickness when transitioning between the first, second and third thicknesses.

In an embodiment where the teat product is a nipple shield, the nipple portion is configured to receive a mother's nipple therein. The nipple portion may be further configured to conform in shape to the mother's nipple.

The nipple shield may be of any suitable size to securely sit in engagement with the mother's breast. In one example, the shield may be between about 6 cm and 10 cm in length or diameter, preferably about 8 cm in length or diameter.

In an embodiment, the nipple portion is substantially dome-shaped. In an embodiment, the nipple portion is substantially conical in shape having a rounded or substantially flat outer end.

In a second aspect, the present invention provides a method for producing a customised teat product for a subject, the method including:
performing a 3D scan of at least a nipple region of the subject's breast to create a 3D model of the nipple region;
creating a 3D product model for a teat product, incorporating a teat that corresponds in shape to at least the nipple region of the subject, based on the 3D model of the nipple region; and
generating a physical teat product based on the 3D product model using an additive or subtractive manufacturing apparatus, wherein the teat product includes a base portion and a nipple portion extending from the base portion, the teat product having a pliable region, wherein said pliable region of the teat product is configured to be deformable in shape in response to force applied by a mouth of a feeding infant.

In an embodiment, the teat product is generated using a 3D printer.

Preferably, the teat product is one of a baby bottle teat, a soother, or a nipple shield.

The nipple region may include at least a nipple and an areola of the subject. Preferably, the subject is a new mother or an expectant mother.

In a preferred embodiment, the teat product is formed from silicone, latex, rubber, or similar. The teat product may be formed of a bio-compatible material. The teat product may be formed of a smart material.

In an embodiment, the method may further include cropping or isolating the nipple region of the 3D scan from the remainder of the 3D scan before creating the 3D product model. Alternatively, said performing the 3D scan of at least the nipple region of the subject's breast may include initially identifying the nipple region, followed by performing the 3D scan. In such embodiments, the 3D scan of substantially only the nipple region can be captured, thereby making it simpler to create the 3D product model without (or with minimal) further processing. Said cropping or isolating the nipple region of the 3D scan from the remainder of the 3D scan may be automated.

It will be appreciated that features disclosed with respect to the first aspect of the invention are also applicable with respect to the second aspect of the invention, including different combinations of features disclosed.

In a third aspect, the present invention provides a system for producing a customised teat for a subject, the system comprising:
3D scanning apparatus adapted to scan at least a nipple region of the subject's breast, generate a 3D nipple model, and output image data representing the 3D nipple model;
a processor having a modelling module with software operable to allow a user to modify the 3D nipple model to create a 3D product model, the processor being adapted to output data representing a three-dimensional product design; and
an additive or subtractive manufacturing apparatus adapted to generate a teat product based on the 3D product model;
wherein the teat product includes a base portion and a nipple portion extending from the base portion, the teat product having a pliable region, wherein said pliable region of the teat product is configured to be deformable in shape in response to force applied by a mouth of a feeding infant.

In an embodiment, the 3D scan apparatus may include a smartphone.

In an embodiment, the 3D scan apparatus may include an application configured to identify said nipple region of the subject's breast. Preferably, said application is configured to enable cropping or isolating of the nipple region from a remainder of a 3D scan before generating the 3D nipple model.

It will be appreciated that features disclosed with respect to the first and second aspects of the invention are also applicable with respect to the third aspect of the invention, including different combinations of features disclosed.

In a fourth aspect, the present invention provides a teat product produced using the method or system of either the second or third aspects of the invention.

It will be appreciated that features disclosed with respect to the first, second and third aspects of the invention are also applicable with respect to the fourth aspect of the invention, including different combinations of features disclosed.

In a fifth aspect, the present invention provides a 3D modelling computing device comprising:
a memory for storing data and a computer program;
a processor coupled to said memory for executing said computer program stored in said memory; and
a modelling application forming part of said computing program, said modelling application including instructions for performing the method steps of:
receiving a 3D nipple model relating to a nipple region of a subject's breast;
modifying the 3D nipple model to create a 3D product model, based on user input, said 3D product model relating to a teat product, wherein the teat product includes a base portion and a nipple portion extending from the base portion, the teat product having a pliable region, wherein said pliable region of the teat product is configured to be deformable in shape in response to force applied by a mouth of a feeding infant.

In a preferred embodiment, the 3D product model is suitable as an input to an additive or subtractive manufacturing apparatus. Preferably, the teat product is selected from the group consisting of: an infant soother, a bottle teat, and a nipple shield. Further preferably, one or more teat product templates are stored in the memory and the step of modifying the 3D nipple model to create a 3D product model comprises applying one of the teat product templates, selected by the user, to the 3D nipple model.

It will be appreciated that features disclosed with respect to the first, second, third and fourth aspects of the invention are also applicable with respect to the fifth aspect of the invention, including different combinations of features disclosed.

In a sixth aspect, the present invention provides an apparatus for implementing any one of the aforementioned methods.

It will be appreciated that features disclosed with respect to the first, second, third, fourth and fifth aspects of the invention are also applicable with respect to the sixth aspect of the invention, including different combinations of features disclosed.

In a seventh aspect, the present invention provides a computer program product including a computer readable medium having recorded thereon a computer program for implementing any one of the methods described above.

It will be appreciated that features disclosed with respect to the first, second, third, fourth, fifth and sixth aspects of the invention are also applicable with respect to the seventh aspect of the invention, including different combinations of features disclosed.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a side view of a nipple shield in accordance with an embodiment of the invention;

FIG. 2 is a top view of the nipple shield of FIG. 1;

FIG. 3 is a flow diagram illustrating a method of producing a customised teat product;

FIG. 4 is a schematic representation of a system on which one or more embodiments of the present disclosure may be practised;

FIG. 5 is a schematic block diagram representation of a system that includes a general purpose computer on which one or more embodiments of the present disclosure may be practised;

FIG. 6 is an upper perspective view of a nipple shield of another embodiment of the invention;

FIG. 7 is a lower perspective view of the nipple shield of FIG. 6;

FIG. 8 is top view of the nipple shield of FIG. 6; and

FIG. 9 is a side cross-section view of the nipple shield of FIG. 6.

### DETAILED DESCRIPTION OF THE EMBODIMENTS

The present disclosure provides a teat product, and a method and system for producing a customised maternity teat product. The term ‘teat product’, as it is used throughout this specification, refers to any product having an imitation nipple component, designed to be used in conjunction with the feeding of an infant. Examples of common teat products include baby bottle teats, soothers/pacifiers, and nipple shields

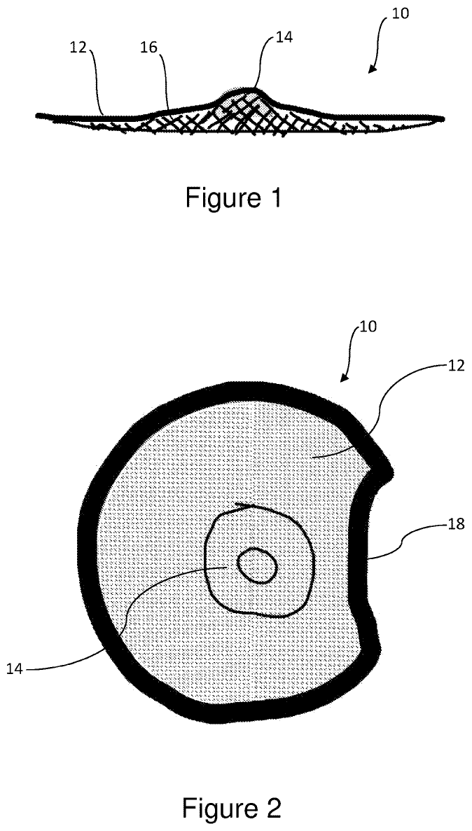

Reference is made to FIGS. 1 and 2, which provides a teat product in the form of a nipple shield 10. Nipple shield 10 includes a substantially circular shaped base portion 12 that is configured to generally wrap around and/or overlie a portion of a mother's breast (e.g. the areola and surrounding portions of the breast), thereby retaining the nipple shield in operative engagement with the mother's breast. Extending outwardly from a generally central region of the base portion 12 is a nipple portion 14 that is configured to generally wrap around and/or overlie the mother's nipple. Nipple portion 14 is substantially conical in shape having a rounded outer end as shown in FIG. 1.

Typically, prior art nipple shields are formed of a material that is somewhat rigid in shape, albeit having some degree of elasticity. Such nipple shields are not particularly deformable in shape in response to force applied by a mouth of a feeding baby, and/or they are generally inflexible. As a result, the baby's feeding experience is generally reduced, as the experience is significantly altered in terms of the baby's sensation when feeding in comparison to direct feeding of the mother's exposed breast. This may also be known as nipple confusion.

In the depicted embodiment, substantially the whole nipple shield 10 is formed of a pliable material, which provides the nipple shield 10 with a degree of deformability and flexibility in shape in response to force (e.g. suction) applied by the mouth of the feeding infant. In the depicted embodiment, nipple shield 10 is formed of a mesh 16, also referred to as meshed region 16. However, in other embodiments, the pliable material is not necessarily a mesh or meshed region, but the pliable material (or a pliable region of the nipple shield) is formed of a different material to the remainder of the nipple shield, or may include an altered region providing said deformability of the nipple shield.

The provision of a nipple shield having a region formed of mesh 16 (in this case the whole nipple shield 10) results in the nipple shield being more readily deformable and flexible in response to force applied by the mouth of the feeding infant. Further, the meshed region 16 of the nipple shield 10 is more responsive due to the improved flexibility and deformability of the meshed region 16. In other words, the meshed region 16 of the nipple shield 10 is configured to have increased stretchability whilst still being resilient, resulting in the meshed region returning back into its original shape. In particular, meshed region 16 of nipple shield 10 is configured to enable elongation and/or deformation of the nipple portion 14. Further, the meshed region 16 is configured to enable nipple shield 10 to substantially conform in shape to a nipple. By being conformable in shape to the nipple of the mother, nipple shield 10 helps reduce nipple confusion by providing the infant with a familiar nipple shape in the infant's mouth (i.e. the mother's nipple). In particular, nipple portion 14 is configured to conform in shape to the nipple when the infant imprints or otherwise impresses on nipple portion 14. As a result of provision of the meshed region, the baby's breastfeeding experience is generally enhanced relative to prior art nipple shields. Further, the meshed region prevents or reduces milk from pooling in the base of the nipple shield, thereby making it a more hygienic and pleasant experience for the mother. Further, the closeness of the nipple to nipple shield 10 can improve nipple stimulation, thereby improving the release of lactation hormones. This can result in improved milk supply to the infant relative to large, non-deformable and more rigid nipple shields.

It will be appreciated that there is no requirement for the whole nipple shield to be formed of a mesh. In other embodiments, the nipple shield may have a region formed of a mesh and a region that is not formed of a mesh. The meshed region may generally enable at least nipple portion 14 to be stretchable relative to base portion 12 as this is the main part of nipple shield 10 that the baby will engage with. Milk (or other fluid) may generally pass through meshed region 16. However, in other embodiments, there may be one or more dedicated openings in nipple portion 14 for fluid to pass through. The meshed region 16 may further include nipple portion 14 and a region surrounding nipple portion 14. For example, the meshed region 16 may be between about 1 cm and 5 cm in diameter, with nipple portion 14 being arranged substantially centrally of the meshed region.

In the case where only a portion of the nipple shield has a meshed region, nipple shield 10 or meshed region 16 may be of any suitable form, e.g. woven or non-woven mesh. In one example, the mesh may be formed of a suitable netting material. In another example, the mesh may be formed by an array of perforations/openings produced in the nipple shield 10. Openings of the mesh may have a diameter between about 0.5 mm and about 3 mm, preferably between 1 mm and 3 mm. It will be appreciated that the size of the openings, along with the density of the number of openings per unit area, in part influence the deformability of the meshed region of the teat product.

With reference to FIG. 2, nipple shield 10 includes a cut-out portion 18 configured to facilitate direct skin contact of the baby with the mother during feeding, e.g. direct skin contact between the baby's cheek and the mother's breast. Cut-out portion 18 is substantially semi-circular in shape. However, the cut-out portion may be of any other suitable shape to facilitate direct skin contact of the baby with the mother during feeding in a manner to enhance to feeding experience for the baby. Further, there may be more than one cut-out portion provided.

Nipple shield 10 may be formed of any suitable material to achieve the desired level of deformability and flexibility. Preferably, nipple shield 10 is formed of a medical grade silicone or similar.

Whilst the depicted embodiment has been described with respect to a nipple shield, it will be appreciated that the teat product may instead be a baby bottle teat or a soother/pacifier.

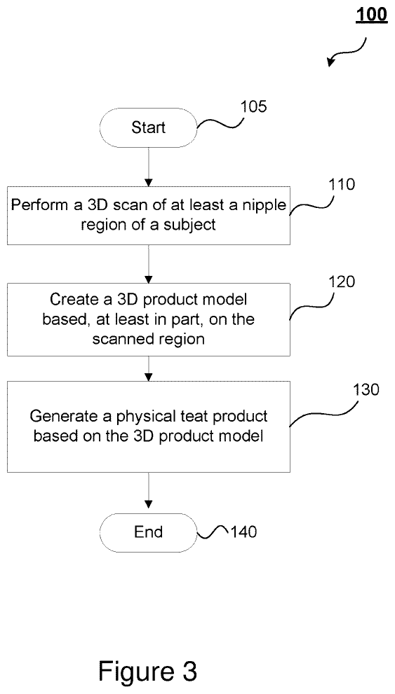

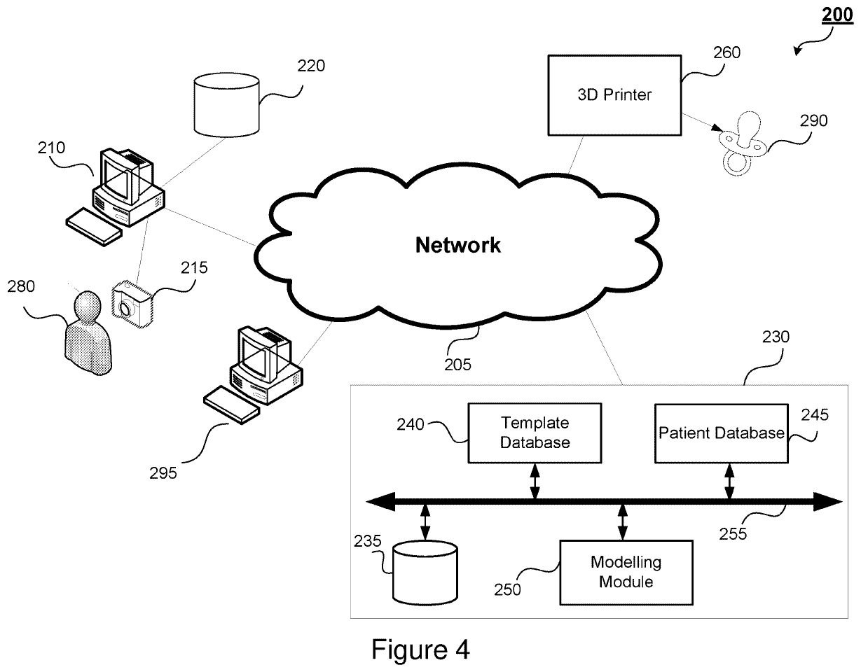

FIG. 3 provides a flow diagram illustrating a method 100 and FIG. 4 depicts a system 200, both the method and system being for producing a customised teat product 290 for a given subject 280, usually a new or expectant mother.

As shown in FIG. 3, the method 100 begins at a Start step 105 and proceeds to step 110, which performs a three-dimensional (3D) scan of at least a portion of a breast of the subject 280, including the nipple region of the breast. Typically, the nipple region scanned will include both the nipple and areola.

As shown in FIG. 4, a 3D scanning device 210, having a camera 215 and a memory 220, is used to collect 3D data on the shape of the nipple region of the breast of the subject 280. The 3D scanner 210 uses the 3D data to create a 3D model of the scanned nipple region, which is stored in the memory 220 as a 3D nipple model. It will be appreciated that the camera 215 and memory 220 may be either integral to the scanning device 210 or external peripherals connected to the scanning device 210. In an embodiment, scanning device 210 may be a smartphone.

3D scanning device 210 may have an application that crops out or isolates the nipple region from the remainder of the collected 3D data, thereby directly creating a 3D model of the scanned nipple region. Said cropping or isolation of the nipple region may be automated.

Returning to FIG. 3, control passes from step 110 to step 120, which uses the 3D model of the scanned nipple region to create a 3D product model of a teat product by altering the 3D model of the scanned nipple region. For example, in one embodiment, the 3D model is modified to create a baby bottle teat by truncating the 3D model, as appropriate, and adding a circumferential flange.

As shown in FIG. 4, the 3D model of the scanned nipple region, or output image data representative of the 3D nipple model, is transmitted from the memory 220 of the scanning device 210 via a network 205 to a modelling computer 230 having a memory 235, a template database 240, a patient database 245, and a modelling module 250, all of which communicate via a bus 255. The 3D model of the scanned nipple region is stored in the patient database 245 and, by way of the modelling module 250, the modelling computer 230 allows a user to create a 3D product model using the 3D model of the scanned nipple region.

A user accesses the modelling computer 230, either directly or by using the scanning device 210 or a computing device 295 coupled to the network 205, in order to provide user input to the modelling module 250. In one embodiment, the user uses an input device of the scanning device 210 or the computing device 295 to transmit user input to the modelling module 250 of the modelling computer 230. The modelling module 250 receives the user input to create the 3D product model.

The template database 240 optionally provides one or more teat product 290 templates, such as a template for the flange of a baby bottle teat, which facilitate the creation of the 3D product model. In an alternative implementation, the scanning device 210 and modelling computer 230 are integrated into a single computing device.

Returning to FIG. 3, control passes from step 120 to step 130, which generates a physical teat product 290 based on the 3D product model by using the cropped 3D model of the scanned nipple region and attaching a base to that model. For example, in one embodiment, a physical teat product 290 is produced from an additive manufacturing device, such as a 3D printer, based on the 3D product model and, for example, using silicone as the printing medium (although other suitable materials may be used). In such an embodiment, the 3D printer lays down successive layers of silicone in accordance with the 3D product model to produce the physical teat product 290. Other additive or subtractive manufacturing techniques, such as continuous liquid interface production (CLIP) may also be employed to produce the physical teat product 290 from the 3D product model.

As depicted in FIG. 4, the 3D product model is transmitted from the modelling computer 230 via the network 205 to an additive manufacturing device 260, which uses the 3D product model to produce the teat product 290. In this example, the teat product 290 is an infant soother/pacifier. However, it will be appreciated that the teat product 290 may be embodied as any teat product. Control passes from step 130 to an End step 140 and the method 100 terminates.

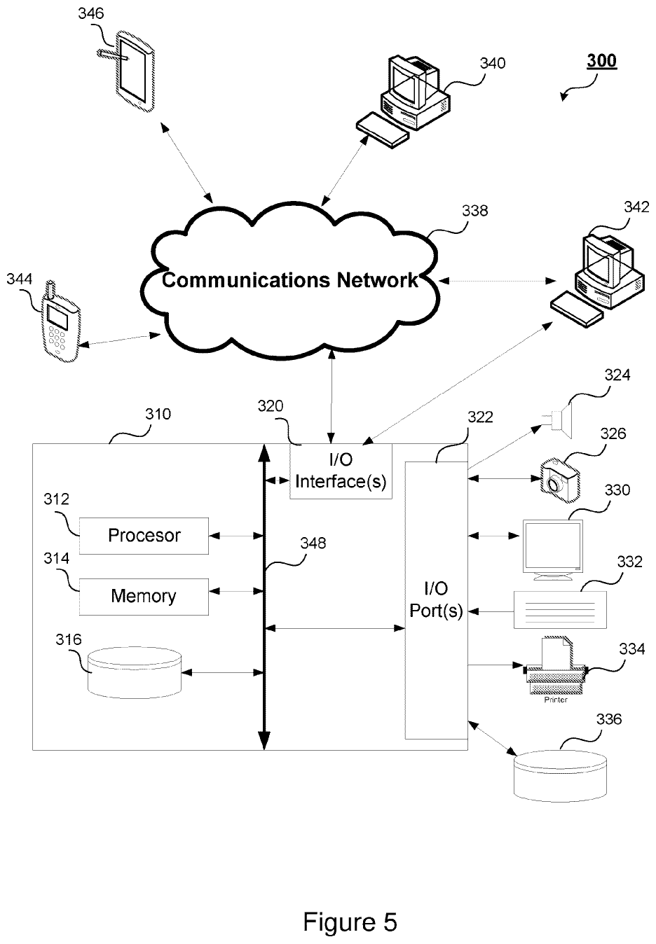

The system for producing a teat product 290 of the present disclosure may be practised using one or more computing devices, such as a general purpose computer or computer server. FIG. 5 is a schematic block diagram of a system 300 that includes a general purpose computer 310. The general purpose computer 310 includes a plurality of components, including: a processor 312, a memory 314, a storage medium 316, input/output (I/O) interfaces 320, and input/output (I/O) ports 322. Components of the general purpose computer 310 generally communicate using one or more buses 348.

The memory 314 may be implemented using Random Access Memory (RAM), Read Only Memory (ROM), or a combination thereof. The storage medium 316 may be implemented as one or more of a hard disk drive, a solid state “flash” drive, an optical disk drive, or other storage means. The storage medium 316 may be utilised to store one or more computer programs, including an operating system, software applications, and data. In one mode of operation, instructions from one or more computer programs stored in the storage medium 316 are loaded into the memory 314 via the bus 348. Instructions loaded into the memory 314 are then made available via the bus 348 or other means for execution by the processor 312 to implement a mode of operation in accordance with the executed instructions.

One or more peripheral devices may be coupled to the general purpose computer 310 via the I/O ports 322. In the example of FIG. 5, the general purpose computer 310 is coupled to each of a speaker 324, a camera 326, a display device 330, an input device 332, a printer 334, and an external storage medium 336. The speaker 324 may be implemented using one or more speakers, such as in a stereo or surround sound system. In the example in which the general purpose computer 310 is utilised to implement the scanning device 210, one or more peripheral devices may relate to the camera 215 and memory 220 connected to the I/O ports 322. In the example in which the general purpose computer 310 is utilised to implement the modelling computer 230, scanning device 210, or computing device 295, one or more peripheral devices may relate to a keyboard, touch tablet, mouse, or other input device for providing user input to the modelling module 250.

The camera 326 may be a webcam, or other still or video digital camera, and may download and upload information to and from the general purpose computer 310 via the I/O ports 322, dependent upon the particular implementation. For example, images recorded by the camera 326 may be uploaded to the storage medium 316 of the general purpose computer 310. Similarly, images stored on the storage medium 316 may be downloaded to a memory or storage medium of the camera 326. The camera 326 may include a lens system, a sensor unit, and a recording medium.

The display device 330 may be a computer monitor, such as a cathode ray tube screen, plasma screen, or liquid crystal display (LCD) screen. The display 330 may receive information from the computer 310 in a conventional manner, wherein the information is presented on the display device 330 for viewing by a user. The display device 330 may optionally be implemented using a touch screen to enable a user to provide input to the general purpose computer 310. The touch screen may be, for example, a capacitive touch screen, a resistive touchscreen, a surface acoustic wave touchscreen, or the like.

The input device 332 may be a keyboard, a mouse, a stylus, drawing tablet, or any combination thereof, for receiving input from a user. The external storage medium 336 may include an external hard disk drive (HDD), an optical drive, a floppy disk drive, a flash drive, or any combination thereof and may be implemented as a single instance or multiple instances of any one or more of those devices. For example, the external storage medium 336 may be implemented as an array of hard disk drives.

The I/O interfaces 320 facilitate the exchange of information between the general purpose computing device 310 and other computing devices. The I/O interfaces may be implemented using an internal or external modem, an Ethernet connection, or the like, to enable coupling to a transmission medium. In the example of FIG. 5, the I/O interfaces 322 are coupled to a communications network 338 and directly to a computing device 342. The computing device 342 is shown as a personal computer, but may be equally practised using a smartphone, laptop, or a tablet device. Direct communication between the general purpose computer 310 and the computing device 342 may be implemented using a wireless or wired transmission link.

The communications network 338 may represent the network 205 of FIG. 4 and may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 310 is able to communicate via the communications network 338 to other computing devices connected to the communications network 338, such as the mobile telephone handset 344, the touchscreen smartphone 346, the personal computer 340, and the computing device 342.

One or more instances of the general purpose computer 310 may be utilised to implement the scanning device 210 and modelling computer 230 to implement a 3D modelling system for producing a customised teat product in accordance with the present disclosure. In such an embodiment, the memory 314 and storage 316 are utilised to store data relating to 3D modelling software, teat product templates, and patient data. Software for implementing the 3D modelling system is stored in one or both of the memory 314 and storage 316 for execution on the processor 312. The software includes computer program code for implementing method steps in accordance with the method of FIG. 3 described herein.

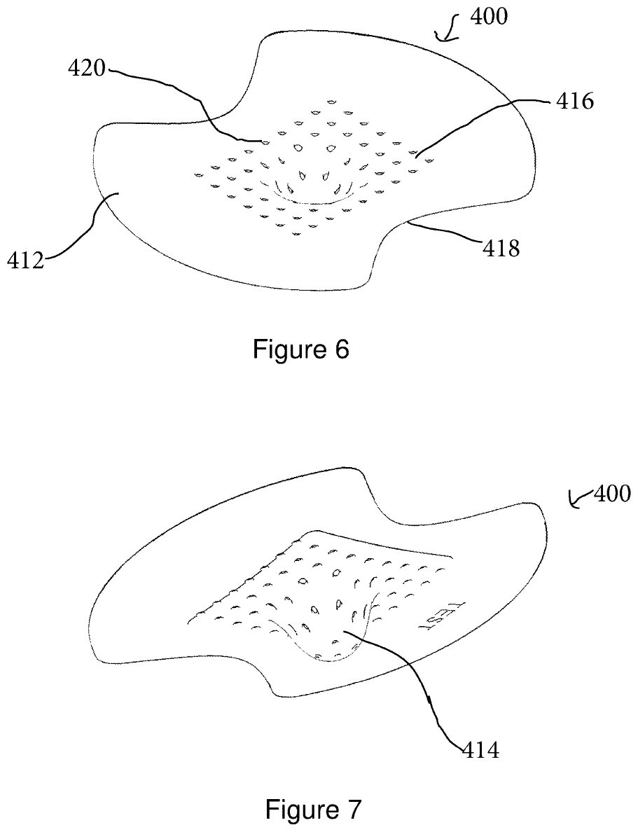

Reference is now made to FIGS. 6-9, which depict a nipple shield 400 in accordance with another embodiment of the invention. It will be appreciated that nipple shield 400 shares various features with that of nipple shield 10, and therefore these features will not be discussed in detail again.

Nipple shield 400 includes a substantially oval shaped base portion 412 that is configured to generally wrap around and/or overlie a portion of a mother's breast (e.g. the areola and surrounding portions of the breast), thereby retaining the nipple shield in operative engagement with the mother's breast. Extending outwardly from a generally central region of the base portion 412 is a nipple portion 414 that is configured to generally wrap around and/or overlie the mother's nipple. Nipple portion 414 is substantially conical in shape having a rounded outer end as best shown in FIG. 7.

In the depicted embodiment, nipple shield 400 includes a pliable region 416 having a plurality of openings 418, which provide the nipple shield 400 with a degree of deformability and flexibility in shape in response to force (e.g. suction) applied by the mouth of the feeding infant. It will be appreciated that pliable region 416 may be considered a meshed region and pliable region 416 provides nipple shield 400 with similar functionality to meshed region 16 of nipple shield 10.

Nipple shield 100 includes two diametrically opposed cut-out portions 418 configured to facilitate direct skin contact of the baby with the mother during feeding, e.g. direct skin contact between the baby's cheek and the mother's breast. Cut-out portions 418 are substantially semi-circular in shape. It will be appreciated that the precise arrangement of the cut-out portions can vary to what is depicted in this embodiment.

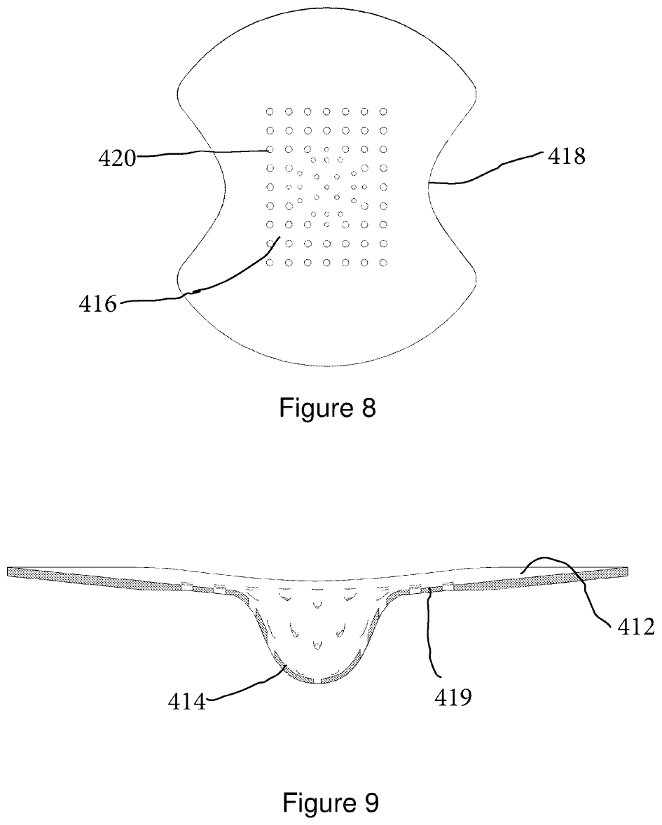

Openings 420 include two different size openings as will be appreciated from FIG. 8. The openings 420 generally positioned in a central portion of the pliable region 416 includes openings of smaller diameter, whereas openings 420 generally positioned peripheral to central portion of the pliable region 416 include openings of larger diameter. It will be appreciated that openings 420 of smaller diameter are provided at nipple portion 414. Openings 420 are generally arranged in an organised pattern, with a plurality of rows/columns of openings arranged about nipple portion 414 to enable elongation and/or deformation of nipple portion 414. The depicted embodiment provides one example of a suitable arrangement of openings. However, other arrangements are also possible.

As will evident from FIG. 9, nipple shield 400 need not have a uniform thickness. In the depicted embodiment, a radially outer portion of base portion 412 includes a first thickness, the nipple portion 414 includes a second thickness, and a transition portion 419, between the radially outer portion of base portion 412 and nipple portion 414, includes a third thickness, wherein the first thickness is about 1 mm in thickness, the second thickness is about 0.7 mm in thickness and the third thickness is about 0.6 mm in thickness. Base portion 412 gradually tapers in thickness radially inwardly towards transition portion 419 and then gradually thickens towards nipple portion 414. The provision of the relatively thinner transition portion 419 provides greater deformability, in this case elongation, of nipple portion 414.

The present disclosure provides a customisable teat product that can be formed of different size and shape to suit the mother of the infant. In the alternative, the teat product can be formed of different standardised size and shape, enabling selection by the mother of a suitable product.

It will be understood that the invention disclosed and defined in this specification extends to all alternative combinations of two or more of the individual features mentioned or evident from the text or drawings. All of these different combinations constitute various alternative aspects of the invention as disclosed in PCT/AU2023/050820, the entire contents of which are incorporated herein by this reference.

What is claimed is:

1. A teat product having a base portion and a nipple portion extending from the base portion, the teat product having a pliable region, wherein said pliable region of the teat product is configured to be deformable in shape in response to force applied by a mouth of a feeding infant.

2. The teat product of claim 1, wherein the nipple portion is disposed in the pliable region.

3. The teat product of claim 1, wherein the nipple portion is arranged substantially centrally of the pliable region.

4. The teat product of claim 1, wherein the nipple portion is arranged substantially centrally of the base portion.

5. The teat product of claim 1, wherein the pliable region is coaxial with the nipple portion.

6. The teat product of claim 1, wherein the pliable region is between about 1 cm and 5 cm in diameter.

7. The teat product of claim 1, wherein the pliable region is configured to enable at least portion of the teat product to substantially conform in shape to a nipple.

8. The teat product of claim 6, wherein the pliable region is configured to enable the nipple portion to substantially conform in shape to a nipple.

9. The teat product of claim 8, wherein the nipple portion is configured to conform in shape to the nipple when the infant imprints or otherwise impresses on the nipple portion.

10. The teat product of claim 1, wherein the pliable region includes a plurality of openings, wherein said plurality of openings are configured to impart said deformability in shape of the pliable region.

11. The teat product of claim 10, wherein the openings have a diameter between about 0.1 mm and about 3 mm.

12. The teat product of claim 1, wherein said pliable region of the teat product is configured to have increased stretchability relative to a remainder of the teat product.

13. The teat product of claim 1, wherein the teat product is substantially formed of a silicone material, latex, rubber, or similar.

14. The teat product of claim 1, wherein the teat product is a baby bottle teat, a soother/pacifier, or a nipple shield.

15. The teat product of claim 1, wherein the teat product is a nipple shield, wherein the nipple shield includes at least one cut-out portion configured to facilitate direct contact of the infant with the mother during feeding.
